(19) United States
(12) Patent Application Publication
(10) Pub. No.: US 2017/0258702 A1
(43) Pub. Date: Sep. 14, 2017

(54) BI-MODAL EMULSIONS

(71) Applicant: DOW CORNING CORPORATION, MIDLAND, MI (US)

(72) Inventor: SAMUEL COSTANZO, MIDLAND, MI (US)

(21) Appl. No.: 15/515,173

(22) PCT Filed: Nov. 30, 2015
(86) PCT No.: PCT/US2015/062887

§ 371 Date: Mar. 29, 2017

Related U.S. Application Data
(60) Provisional application No. 62/087,013, filed on Dec. 3, 2014.

(51) Int. Cl. A61K 8/898; A61K 8/891; A61Q 19/00; A61K 8/06

(52) U.S. Cl. CPC A61K 8/898; A61K 8/062; A61K 8/891; A61Q 19/00; A61K 2800/10; A61K 2800/594

(57) ABSTRACT

The present invention provides a process for making a bi-modal water continuous emulsion comprising forming a mixture comprising 100 parts by weight of a hydrophobic oil and 1 to 1000 part by weight of a water continuous emulsion having at least one surfactant, and admixing additional quantities of the water continuous emulsion and/or water to the mixture to form a bi-modal water continuous emulsion, wherein the water continuous emulsion forms a first dispersed phase of particle size P1 and the hydrophobic oil forms a second dispersed phase of particle size P2, and wherein the ratio P2:P1 is less than 1. The present invention provides bi-modal water continuous emulsion having a first dispersed phase of particle size P1 and a second dispersed phase of particle size P2 wherein the ratio P2:P1 is less than 1, and personal care and coating compositions comprising the bi-modal water continuous emulsion.

### CROSS-REFERENCE TO RELATED APPLICATIONS

This application claims the benefit of U.S. Provisional Patent Application No. 62/087,013 filed Dec. 3, 2014.

### FIELD OF THE INVENTION

The present invention is in the field of emulsions and processes for making emulsions. More particularly, the present invention is in the field of bi-modal water continuous emulsions and processes for making bi-modal water continuous emulsions.

### BACKGROUND OF THE INVENTION

While numerous advancements have been made in the emulsions field, there are several long standing needs that remain. For example, as the percent solids of an emulsion increases, in most emulsions the viscosity also increases. Emulsions having a solids level greater than 75 weight % can become so viscous that they are non-pourable. This effectively renders such emulsion products unusable in many applications due to the handling difficulties of such viscous compositions.

Another long standing need in this field is to stabilize emulsions with a minimal amount of surfactants. This is a particular need when the emulsions are used to form coatings, such as protective architectural coatings. Residual surfactant on coatings formed from emulsions can have several detrimental effects on the physical property profile of the coatings such as decreased hydrophobicity and/or poorer dirt resistance. The use of emulsions with minimal amount of surfactants is also highly desirable for application in personal care products, especially for skin and cosmetic formulations where residual surfactants may cause skin irritation.

Reducing the presence of solvents, un-reacted siloxanes, catalyst residues, cyclic polymerization byproducts, and other impurities in silicone emulsions is an ongoing challenge in the art. The need to reduce such impurities may arise, among other reasons, when such impurities are incompatible with downstream applications (for example, medical, cosmetic, and personal care applications), where the presence of such impurities would reduce the stability of an emulsion, or where regulatory requirements require removal or reduction of their presence. In particular, there is an interest to reduce the presence of cyclosiloxanes, such as octamethylcyclotetrasiloxanes and decamethylcyclopentasiloxanes, in silicone emulsions.

Thus, a need still exists for processes that provide emulsion products having high solids contents that remain pourable. A further need exists to reduce the amount of surfactants in emulsion products, especially at high solid content emulsions. Yet, a further need exists to provide silicone emulsions having reduced content of cyclosiloxane concentrations.

### SUMMARY OF THE INVENTION

The present invention provides high solids content emulsions having lower viscosities than emulsions of similar solids content prepared by other methods. The present invention relates to a process for preparing bi-modal water continuous emulsions, that is, water continuous emulsions containing at least two distinct dispersed phases.

In one embodiment, the present invention provides a process for making a bi-modal water continuous emulsion comprising:
I) forming a mixture comprising:
   A) 100 parts by weight of a hydrophobic oil, and
   B) 1 to 1000 part by weight of a water continuous emulsion having at least one surfactant;
II) admixing additional quantities of the water continuous emulsion and/or water to the mixture from step I) to form a bi-modal water continuous emulsion,
   wherein the water continuous emulsion forms a first dispersed phase of particle size P1 and the hydrophobic oil forms a second dispersed phase of particle size P2, and wherein the ratio P2:P1 is less than 1.

The processes and emulsions of the present invention provides advantages, for example, versatility to prepare a wide range of bi-modal water continuous emulsions having high solids content. The processes of the present invention may be used to prepare a variety of bi-modal water continuous emulsions having two distinct dispersed phases. Each distinct dispersed phase may contain either an organic oil or a silicone oil.

### DETAILED DESCRIPTION OF THE INVENTION

All amounts, ratios, and percentages are by weight unless otherwise indicated.

The articles ‘a’, ‘an’, and ‘the’ each refers to one or more, unless otherwise indicated by the context of the specification.

The disclosure of ranges includes the range itself and also anything subsumed therein, as well as endpoints. For example, disclosure of a range of 2.0 to 4.0 includes not only the range of 2.0 to 4.0, but also 2.1, 2.3, 3.4, 3.5, and 4.0 individually, as well as any other number subsumed in the range. Furthermore, disclosure of a range of, for example, 2.0 to 4.0 includes the subsets of, for example, 2.1 to 3.5, 2.3 to 3.4, 2.6 to 3.7, and 3.8 to 4.0, as well as any other subset subsumed in the range.

Similarly, the disclosure of Markush groups includes the entire group and also any individual members and subgroups subsumed therein. For example, disclosure of the Markush group a hydrogen atom, an alkyl group, an aryl group, an aralkyl group, or an alkaryl group includes the member alkyl individually; the subgroup alkyl and aryl; and any other individual member and subgroup subsumed therein.

For U.S. practice, all patent application publications and patents referenced herein, or a portion thereof if only the portion is referenced, are hereby incorporated herein by reference to the extent that incorporated subject matter does not conflict with the present description, which would control in any such conflict.

The term “alternatively” indicates a different and distinct embodiment.

The term “comprises” and its variants (comprising, comprised of) are open ended.

The term “consists of” and its variants (consisting of) are closed ended.

The term “may” confers a choice, not an imperative.

The term “optionally” means is absent, or alternatively is present.

The present invention provides a process for making a bi-modal water continuous emulsion comprising:
I) forming a mixture comprising:
   A) 100 parts by weight of a hydrophobic oil, and
   B) 1 to 1000 part by weight of a water continuous emulsion having at least one surfactant;
II) admixing additional quantities of the water continuous emulsion and/or water to the mixture from step I) to form a bi-modal water continuous emulsion,
   wherein the water continuous emulsion forms a first dispersed phase of particle size P1 and the hydrophobic oil forms a second dispersed phase of particle size P2, and wherein the ratio P2:P1 is less than 1.

In one embodiment, the mixture of step I) consists essentially of components A) and B). In another embodiment, first dispersed phase and the second dispersed phase comprise at least 70 weight percent of the bi-modal water continuous emulsion. The quantity of the water continuous emulsion and/or water added to the mixture is such so as to provide a bi-modal water continuous emulsion containing at least 70% by weight of components A) and B).

The hydrophobic oil may be a silicone, such as an amino-functionalized silicone, or organic oil. The water continuous emulsion may be a silicone emulsion or an organic emulsion. The silicone emulsion may comprises a silicone that is a product of a hydrosilylation reaction.

In another embodiment, the invention provides a bi-modal water continuous emulsion produced by the processes of the present invention. The bi-modal water continuous emulsion comprises an hydrophobic oil as the first dispersed phase and an amino-functionalized silicone as the second dispersed phase, wherein the first dispersed phase has a particle size P1 and the second dispersed phase has a particle size P2, and wherein the ratio P2:P1 is less than 1. The hydrophobic oil may be a silicone that is a product of a hydrosilylation reaction.

The bi-modal water continuous emulsions are water continuous emulsions having two distinct dispersed phases. As used herein, “dispersed phase” refers to the water insoluble particles suspended in the continuous aqueous phase of the emulsions. In one embodiment, the first dispersed phase contains a hydrophobic oil, which may be either an organic oil or a silicone. In another embodiment, the second dispersed phase contains a silicone that is provided from a previously formed water continuous emulsion. Each dispersed phase may be characterized by its own average particle size distribution in the bi-modal water continuous emulsions. In other words, the average particle size of the two distinct dispersed phases demonstrate a “bi-modal” distribution.

The first step in the present process is to form a mixture comprising of, consisting essentially of, or consisting of:
A) 100 parts by weight of a hydrophobic oil, and
B) 1 to 1000 parts by weight of a water continuous emulsion having at least one surfactant.

### The Hydrophobic Oil

The bi-modal water continuous emulsions contain a second dispersed phase containing a hydrophobic oil (designated herein as component (A)). The hydrophobic oil (A) may be one or more than one hydrophobic oil which may be the same or different hydrophobic oils. The hydrophobic oil (A) in the second dispersed phase need not be pre-emulsified. In other words, the hydrophobic oil can be derived from a neat or non-emulsified hydrophobic oil. The hydrophobic oil (A) may be selected from a) an organic oil, b) a silicone, or any mixtures or combinations thereof.

In one embodiment, the second dispersed phase may be an organic oil phase. The organic oil may comprise an organic compound or an organic polymer. The organic oil may be selected from hydrocarbons, esters, oils derived from natural fats or oils, organic polymers, or mixtures thereof.

Examples of suitable organic oil components include, but are not limited to, natural oils such as coconut oil; hydrocarbons such as mineral oil, paraffins and hydrogenated polyisobutene; fatty alcohols such as octyldodecanol; esters such as C12-C15 alkyl benzoate; diesters such as propylene dipelargonate; and triesters, such as glyceryl trioctanoate.

Examples of esters as suitable organic oil may have the structure QCO-OQ′ wherein QCO represents the carboxylic acid radical and wherein OQ′ is an alcohol residue. Examples of these esters include, but are not limited to, isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or mixtures of octyldodecanol, acetylated lanolin alcohol, cetyl acetate, isododecanol, polyglyceryl-3-diisostearate, are mixtures thereof.

Examples of natural oils include, but are not limited to, castor oil, lanolin and lanolin derivatives, triisocetyl citrate, sorbitan sesquioleate, C10-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, castor oil, illipe butter, rapeseed oil, soybean oil, sunflower seed oil, pine oil, tallow, tricaprin, trihydroxystearin, triisostearin, trilaurin, trilinolein, trimyristin, triolein, tripalmitin, tristearin, walnut oil, wheat germ oil, cholesterol, and mixtures thereof.

The organic oil may comprise an organic polymer such as, but not limited to, polybutenes or polyisobutylenes, polyacrylates, polystyrenes, polybutadienes, polyamides, polyesters, polyacrylates, polyurethanes, polysulfones, polysulf ides, epoxy functional polymers, as well as copolymers or terpolymers containing these organic polymers, and mixtures of any of these.

Further suitable organic oils may be solid or liquid at room temperature, such as organic butters and organic waxes. Examples of butters include, but are not limited to, cocoa butter, shea butter and mango butter. Examples of organic waxes include, but are not limited to, those selected from synthetic and natural origins such as mineral waxes, animal waxes, vegetal waxes, hydrogenated oils, fatty esters and glycerides which are solid at 25° C. Additional examples of organic waxes include, but are not limited to, esters derived from a monovalent saturated C16-C60 alkanol and a saturated C8-C36 monocarboxylic acid, glycerol triesters of saturated linear C18-C40 carboxylic acids, candelilla wax, carnauba wax, beeswax, saturated linear C16-C18, C20, and C22-C40 carboxylic acids, hardened castor oil, ozokerite, polyethylene wax, microcrystalline wax, ceresin, lanolin wax, rice bran wax, montan wax, orange wax, lemon wax and paraffin wax.

In another embodiment, the hydrophobic oil (A) may be silicone polymers. In this embodiment, the hydrophobic oil phase is considered to be a silicone oil phase. As used herein, a “silicone polymer” refers to a composition containing at least one organopolysiloxane.

Organopolysiloxanes are polymers containing siloxy units independently selected from (R₃SiO_{1/2}), (R₂SiO_{2/2}), (RSiO_{3/2}), or (SiO_{4/2}) siloxy units, where R may be an organic group, alternatively R may be a hydrocarbon group containing 1 to 30 carbons, alternatively R may be an alkyl group containing 1 to 12 carbon atoms, or alternatively R may be methyl or phenyl. These siloxy units are commonly referred to as M, D, T, and Q units respectively. Their molecular structures are listed below:

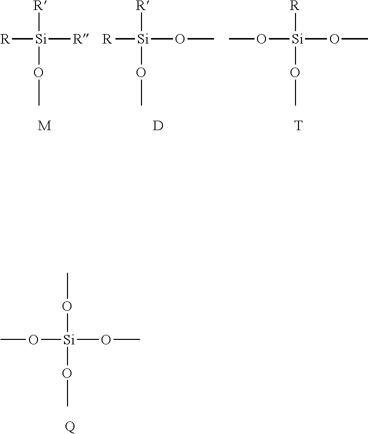

wherein R′ and R″ have the same meaning as R. The siloxy units can be combined in different sequences and amounts to form cyclic, linear, or branched structures. The chemical and physical properties of the resulting polymeric structures vary depending on the number and type of the siloxy units in the organopolysiloxane.

The silicone polymer may contain a single organopolysiloxane, or mixture of various organopolysiloxanes. In some embodiments, the mixture of organopolysiloxanes can react with each other to form higher molecular weight organopolysiloxanes. Such reactions are exemplified by condensation or hydrosilylation reactions.

The silicone polymer may contain silicone fluids, silicone gums, silicone rubbers, silicone elastomers, silicone resins, silicone waxes, saccharide-siloxane polymer, vinyl polymer grafted with a carbosiloxane dendrimers or any combinations thereof.

The organopolysiloxane may be a trimethylsiloxy or hydroxy (SiOH) terminated polydimethylsiloxane. Trimethylsiloxy end blocked polydimethysiloxanes may have the formula Me₃SiO(Me₂SiO_{2/2})_{dp}SiMe₃ wherein the degree of polymerization (dp) is greater than 1, or alternatively the dp is sufficient to provide a kinematic viscosity that may range from 1 to 1,000,000 mm²/s (10⁻⁶ m²/s) at 25° C., or alternatively from 100 to 600,000 mm²/s (10⁻⁶ m²/s) at 25° C., or alternatively from 1000 to 600,000 mm²/s (10⁻⁶ m²/s) at 25° C.

When the silicone polymer contains organopolysiloxanes that can react via hydrosilylation, the silicone polymer contains:
b¹) an organopolysiloxane having at least two silicon-bonded alkenyl groups per molecule,
b²) an organohydrogensiloxane having at least two SiH groups per molecule, and
b³) a hydrosilylation catalyst.

The organopolysiloxane having at least two silicon-bonded alkenyl groups per molecule b¹) comprises at least two siloxy units represented by the formula:

R²RₘSiO_{(4-1-m)/2}

wherein R is as defined above or a hydrocarbon group containing 1 to 30 carbon atoms, R² is an alkenyl group containing 2 to 12 carbon atoms, and m is zero to 2. The R² alkenyl groups of Component b¹) may be exemplified by vinyl, allyl, 3-butenyl, 4-pentenyl, 5-hexenyl, 6-heptenyl, 7-octenyl, 8-nonenyl, 9-decenyl, 10-undecenyl, 4,7-octadienyl, 5,8-nonadienyl, 5,9-decadienyl, 6, 11-dodecadienyl and 4,8-nonadienyl.

The R² alkenyl group may be present on any mono, di, or tri siloxy unit in the organopolysiloxane, for example, (R²R₂SiO_{1/2}), (R²RSiO_{2/2}), or (R²SiO_{3/2}), as well as in combination with other siloxy units not containing an R² substituent, such as (R₃SiO_{1/2}), (R₂SiO_{2/2}), (RSiO_{3/2}), or (SiO_{4/2}) siloxy units where R is as defined above or is a hydrocarbon containing 1 to 30 carbons, alternatively an alkyl group containing 1 to 12 carbons, alternatively an alkyl group containing 1 to 6 carbons or alternatively methyl, provided there are at least two R² substituents in the organopolysiloxane. The monovalent hydrocarbon group R having from 1 to 30 carbon atoms is exemplified by alkyl groups such as: methyl, ethyl, propyl, butyl, hexyl, octyl, and decyl; cycloaliphatic groups such as cyclohexyl; aryl groups such as phenyl, tolyl, and xylyl; and aralkyl groups such as benzyl and phenylethyl.

Component b¹) may be selected from trimethylsiloxy-terminated polydimethylsiloxane-polymethylvinylsiloxane copolymers, vinyldimethylsiloxy-terminated polydimethylsiloxane-polymethylvinylsiloxane copolymers, trimethylsiloxy-terminated polydimethylsiloxane-polymethylhexenylsiloxane copolymers, hexenyldimethylsiloxy-terminated polydimethylsiloxane-polymethylhexenylsiloxane copolymers, trimethylsiloxy-terminated polymethylvinylsiloxane polymers, trimethylsiloxy-terminated polymethylhexenylsiloxane polymers, vinyldimethylsiloxy-terminated polydimethylsiloxane polymers, hexenyldimethylsiloxy-terminated polydimethylsiloxane polymers, or any combination thereof, each having a degree of polymerization of from 10 to 300, or alternatively having a viscosity at 25° C. of from 10 to 1000 mPa·s (10⁻³ Pa·s), alternatively 3 to 1000 mPa·s (10⁻³ Pa·s), or alternatively 10 to 500 mPa·s (10⁻³ Pa·s).

Component b²) is an organohydrogensiloxane having an average of greater than two silicon bonded hydrogen atoms per molecule. As used herein, an organohydrogensiloxane is any organopolysiloxane containing silicon-bonded hydrogen atoms (SiH).

Organohydrogensiloxanes are organopolysiloxanes having at least one SiH containing siloxy unit, that is at least one siloxy unit in the organopolysiloxane has the formula (R₂HSiO_{1/2}), (RHSiO_{2/2}), or (HSiO_{3/2}). Thus, the organohydrogensiloxanes useful in the present invention may comprise any number of (R₃SiO_{1/2}), (R₂SiO_{2/2}), (RSiO_{3/2}), (R₂HSiO_{1/2}), (RHSiO_{2/2}), (HSiO_{3/2}) or (SiO_{4/2}) siloxy units, provided there are on average at least two SiH siloxy units in the molecule. Component b²) can be a single linear or branched organohydrogensiloxane or a combination comprising two or more linear or branched organohydrogensiloxanes that differ in at least one of the following properties: structure, viscosity, average molecular weight, siloxane units, and sequence. There are no particular restrictions on the molecular weight of the organohydrogensiloxane, but typically the viscosity of the organohydrogensiloxane at 25° C. may be from 3 to 10,000 mPa·s (10⁻³ Pa·s), alternatively 3 to 1,000 mPa·s (10⁻³ Pa·s), or alternatively 10 to 500 mPa·s (10⁻³ Pa·s).

The amount of SiH units present in the organohydrogensiloxane may vary, provided there are at least two SiH units per organohydrogensiloxane molecule. The amount of SiH units present in the organohydrogensiloxane is expressed herein as percent SiH which is the weight percent of hydrogen in the organohydrogensiloxane. Typically, the percent SiH varies from 0.01% to 10%, alternatively from 0.1% to 5%, or alternatively from 0.5% to 2%.

The organohydrogensiloxane may comprise the average formula:

(R³₃SiO_{1/2})ₐ(R⁴₂SiO_{2/2})_{b}(R⁴HSiO_{2/2})_{c}

wherein
R³ is hydrogen or R⁴,
R⁴ is a monovalent hydrocarbon group having from 1 to 10 carbon atoms
a≧2,
b≧0, alternatively b=1 to 500, alternatively b=1 to 200, and
c≧2, alternatively c=2 to 200, alternatively c=2 to 100.

The R⁴ may be a substituted or unsubstituted monovalent aliphatic or aromatic hydrocarbyl. Monovalent unsubstituted aliphatic hydrocarbyls are exemplified by, but not limited to, alkyl groups such as methyl, ethyl, propyl, pentyl, octyl, undecyl, and octadecyl and cycloalkyl groups such as cyclohexyl. Monovalent substituted aliphatic hydrocarbyls are exemplified by, but not limited to, halogenated alkyl groups such as chloromethyl, 3-chloropropyl, and 3,3,3-trifluoropropyl. The aromatic hydrocarbyl group is exemplified by, but not limited to, phenyl, tolyl, xylyl, benzyl, styryl, and 2-phenylethyl. In some embodiments, the organohydrogensiloxane may be an SiH-terminated polydimethylsiloxane polymer.

The amounts of components b¹) and b²) used may vary, but typically the amounts of components b¹) and b²) are selected so as to provide a molar ratio of the alkenyl groups to SiH groups that is greater than 1.

Component b³) is a hydrosilylation catalyst. The hydrosilylation catalyst may be any suitable Group VIII metal based catalyst selected from a platinum, rhodium, iridium, palladium or ruthenium. Group VIII group metal containing catalysts useful to catalyze curing can be any of those known to catalyze reactions of silicon bonded hydrogen atoms with silicon bonded unsaturated hydrocarbon groups. The typical Group VIII metal for use as a catalyst to effect cure by hydrosilylation may be a platinum based catalyst. Some typical platinum based hydrosilylation catalysts for curing may be platinum metal, platinum compounds and platinum complexes. Suitable platinum catalysts are described in U.S. Pat. No. 2,823,218 (commonly referred to as “Speier's catalyst) and U.S. Pat. No. 3,923,705. The platinum catalyst may be “Karstedt's catalyst”, which is described in Karstedt's U.S. Pat. No. 3,715,334 and U.S. Pat. No. 3,814,730. Karstedt's catalyst is a platinum divinyl tetramethyl disiloxane complex typically containing about one-weight percent of platinum in a solvent such as toluene. Alternatively the platinum catalyst may be a reaction product of chloroplatinic acid and an organosilicon compound containing terminal aliphatic unsaturation, as described in U.S. Pat. No. 3,419,593. Alternatively, the hydrosilylation catalyst may be a neutralized complex of platinum chloride and divinyl tetramethyl disiloxane, as described in U.S. Pat. No. 5,175,325.

The amounts of catalyst b³) used may vary, but typically an amount is used to effect the hydrosilylation reaction. When the catalyst is a Pt compound, typically a sufficient amount of the compound is added to provide 2 to 500 ppm of Pt in the reaction composition.

Additional components may be added to the hydrosilylation reaction. For example, heptamethyltrisiloxysilane may be added as an endblocker to control molecular weight of the organopolysiloxane product.

When the silicone polymer contains organopolysiloxanes components that can react via condensation, the silicone polymer comprises an organopolysiloxane having at least two siloxy units with a substituent capable of reacting via condensation. Suitable substitutes on the siloxy units of the organopolysiloxanes include silanol, alkoxy, acetoxy, and oxime functional groups. The silicone polymer may further contain a catalyst known in the art for enhancing condensation cure of the organopolysiloxanes such as a tin or titanium catalyst. The organopolysiloxane may be a silanol endblocked polydimethylsiloxane having a kinematic viscosity that may range from 1 to 100,000 mm²/s (10⁻⁶ m²/s) at 25° C., or alternatively from 1 to 10,000 mm²/s (10⁻⁶ m²/s) at 25° C.

The silicone polymer composition may contain organopolysiloxanes having at least one siloxy unit substituted with an organofunctional group. The organofunctional organopolysiloxanes may be characterized by having at least one of the R groups in the formula RₙSiO_{(4-n)/2} be an organofunctional group. Representative non-limiting organofunctional groups include amino, amido, epoxy, mercapto, polyether (polyoxyalkylene) groups, and any mixture thereof. Further examples of organofunctional organopolysiloxanes include those having alkoxylated groups; hydroxyl groups such as the polyorganosiloxanes containing a hydroxyalkyl function, as described in EP1081272, U.S. Pat. No. 6,171,515 and U.S. Pat. No. 6,136,215; bis-hydroxy/methoxy amodimethicone; amino-acid functional siloxanes obtained by reacting an amino acid derivative selected from the group of an N-acyl amino acid and an N-aroyl amino acid with an amino functional siloxane, further described in WO2007/141565; quaternary ammonium functional silicones, described in U.S. Pat. No. 6,482,969 and U.S. Pat. No. 6,607,717, such as Silicone Quaternium-16 (CTFA designation); hydrocarbyl functional organopolysiloxanes comprising a siloxy unit of the formula R⁵R′ᵢSiO_{(3-i)/2} wherein R′ is any monovalent hydrocarbon group, but typically is an alkyl, cycloalkyl, alkenyl, alkaryl, aralkyl, or aryl group containing 1-20 carbon atoms, R⁵ is a hydrocarbyl group having the formula —R⁶OCH₂CH₂OH, wherein R⁶ is a divalent hydrocarbon group containing 2 to 6 carbon atoms and i has a value of from zero to 2, such as described in U.S. Pat. No. 2,823,218, U.S. Pat. No. 5,486,566, U.S. Pat. No. 6,060,044 and U.S. Pat. No. 2,002,0524 (CTFA bis-hydroxyethoxypropyl dimethicone); siloxane-based polyamide such as described in U.S. Pat. No. 6,051,216; silicone polyether-amide block copolymers such as described in US2008/0045687.

In another embodiment, the silicone polymer comprises organopolysiloxanes having amino and polyether functionalities. The silicone polymer may be an amino-terminated organopolysiloxane-polyether block copolymer. For example, the coploymer may be bis-diisopropanolamino-PG-propyl dimethicone/bis-isobutyl PEG-14 copolymer.

The organofunctional group may be present on any siloxy unit having an R substituent, that is, they may be present on any (R₃SiO_{1/2}), (R₂SiO), or (RSiO_{3/2}) unit.

The organofunctional group may be an amino-functional hydrocarbon group. Amino-functional hydrocarbon groups may be designated in the formulas herein as R^{N} and is illustrated by groups having the formula:

—R⁸NHR⁹, —R⁸NR⁹₂, or —R⁸NHR⁸NHR⁹,

wherein each R⁸ is independently a divalent hydrocarbon group having at least 2 carbon atoms, and each R⁹ is independently hydrogen or an alkyl group. Each R⁸ is typically an alkylene group having from 2 to 20 carbon atoms. Some examples of suitable amino-functional hydrocarbon groups are: —CH₂CH₂NH₂, —CH₂CH₂CH₂NH₂, —CH₂CHCH₃NH, —CH₂CH₂CH₂CH₂NH₂, —CH₂CH₂CH₂CH₂CH₂NH₂, —CH₂CH₂CH₂CH₂CH₂CH₂NH₂, —CH₂CH₂NHCH₃, —CH₂CH₂CH₂NHCH₃, —CH₂(CH₃)CHCH₂NHCH₃, —CH₂CH₂CH₂CH₂NHCH₃, —CH₂CH₂NHCH₂CH₂NH₂, —CH₂CH₂CH₂NHCH₂CH₂NH₂, —CH₂CH₂CH₂NHCH₂CH₂CH₂NH₂, —CH₂CH₂CH₂CH₂NHCH₂CH₂CH₂CH₂NH₂, —CH₂CH₂NHCH₂CH₂NHCH₃, —CH₂CH₂CH₂NHCH₂CH₂CH₂NHCH₃, —CH₂CH₂CH₂CH₂NHCH₂CH₂CH₂CH₂NHCH₃, and —CH₂CH₂NHCH₂CH₂NHCH₂CH₂CH₂CH₃.

Examples of silicone resins include, but are not limited to, trimethylsilylsilicate (MQ resin), silsesquioxane resins (T resin), MQ-T resins, and silsesquioxane resin waxes.

A trimethylsilylsilicate (MQ resin) may comprise ≧80 mole % of siloxy units selected from (R¹⁰₃SiO_{1/2})ₐ and (SiO_{4/2})_{d} units, where R¹⁰ is an alkyl group having from 1 to 8 carbon atoms, an aryl group, a carbinol group, or an amino group, with the proviso that ≧95 mole % of the R¹⁰ groups are alkyl groups, a and d>0, and the ratio of a/d=0.5 to 1.5.

MQ resins may contain D and T units, provided that ≧80 mole %, alternatively ≧90 mole % of the total siloxane units are M and Q units. The MQ resins may also contain hydroxy groups. Typically, the MQ resins have a total weight % hydroxy content of 2 to 10 weight %, alternatively 2 to 5 weight %. The MQ resins can also be further “capped” wherein residual hydroxy groups are reacted further with M groups.

A silsesquioxane resins (T resin) may comprise ≧30 mole % of R¹⁰SiO_{3/2} units, where R¹⁰ is as defined above. When ≧40 mole % of the R¹⁰ groups are propyl, the T resin may be named a propyl silsesquioxane resin.

T resins may contain M, D, and Q units, provided that ≧30 mole %, alternatively ≧80 mole %, alternatively ≧90 mole % of the total siloxane units are T units. The T resins may also contain hydroxy and/or alkoxy groups. Typically, the T resins have a total weight % hydroxy content of 2 to 10 weight % and a total weight % alkoxy content 20 weight %; alternatively 6 to 8 weight % hydroxy content and 10 weight % alkoxy content.

MQ and T organopolysiloxane resins may be used alone or combined together.

A MQ-T resin may have the formula (R¹¹₃SiO_{1/2})ₐ (R¹²₂SiO_{2/2})_{b} (R¹³SiO_{3/2})_{c} (SiO_{4/2})_{d} with R¹¹, R¹² and R¹³ independently represent an alkyl group containing from 1 to 8 carbon atoms, an aryl group, a carbinol group or an amino group, where 0.05≦a≦0.5; 0≦b≦0.3; c>0; 0.05≦d≦0.6, and a+b+c+d=1, with the proviso that ≧40 mole % of the R¹³ groups in the siloxane resin are propyl. Representatives of such MQ-T resins are taught in WO2005/075542.

A silsesquioxane resin wax may comprise at least 40 mole % of siloxy units having the formula (R¹⁰₂R¹⁴SiO_{1/2})ₓ(R¹⁵SiO_{3/2})_{y}, where x and y have a value of 0.05 to 0.95, R¹⁰ is as described above, R¹⁴ is a monovalent hydrocarbon having 9-40 carbon atoms, and R¹⁵ is a monovalent hydrocarbon group having 1 to 8 carbon atoms or an aryl group. The R¹⁴ and the ratio of y/x are selected such that the silsesquioxane resin wax has a melting point of 30° C. Representatives of such silsesquioxane resin waxes are described in U.S. Pat. No. 7,482,419.

Examples of silicone waxes include, but are not limited to, C30-45 alkyl methicone and C30-45 olefin (MP>60° C.), Bis-PEG-18 methyl ethyl dimethyl silane, stearyl dimethicone.

Silicone elastomers are a type of tri-dimensional crosslinked silicone polymers. Examples of silicone elastomers include, but are not limited to, those obtained from the crosslinking hydrosilylation reaction of an organohydrogenpolysiloxane with another polysiloxane containing an unsaturated hydrocarbon substituent, such as a vinyl functional polysiloxane, or by crosslinking an organohydrogenpolysiloxane with a hydrocarbon diene or with a terminally unsaturated polyoxyalkylene. Representative examples of such silicone elastomers are described in U.S. Pat. No. 5,880,210 and U.S. Pat. No. 5,760,116. Other examples include silicone elastomers to which organofunctional groups have been grafted onto the silicone organic elastomer backbone, such as alkyls, polyether, and amines. Representative examples of such organofunctional silicone elastomers are described in U.S. Pat. No. 5,811,487, U.S. Pat. No. 5,880,210, U.S. Pat. No. 6,200,581, U.S. Pat. No. 5,236,986, U.S. Pat. No. 6,331,604, U.S. Pat. No. 6,262,170, U.S. Pat. No. 6,531,540, and U.S. Pat. No. 6,365,670, WO2004/104013 and WO2004/103326.

Examples of saccharide-siloxane polymer include, but are not limited to, the reaction product of a functionalized organosiloxane polymer and at least one hydroxy-functional saccharide component comprising 5 to 12 carbon atoms, in such a way that the organosiloxane component is covalently linked via a linking group to the saccharide component. Saccharide-siloxane polymers may be linear or branched. Further examples of saccharide-siloxane polymers are described in US20080199417, US20100105582, WO2012027073, and WO2012027143.

Examples of vinyl polymer grafted with a carbosiloxane dendrimers include, but are not limited to, the reaction product of a vinyl polymer with at least one carbosiloxane dendrimer-based unit. The term “carbosiloxane dendrimer structure” designates a structure with branched groups of high molecular masses with high regularity in the radial direction starting from the simple backbone. Such carbosiloxane dendrimer structures are described in the form of a highly branched siloxane-silalkylene copolymer, for example, in the laid-open Japanese patent application Kokai 9-171154. Other vinyl polymers grafted with a carbosiloxane dendrimer are described in EP0963751.

The silicone in the first and/or second dispersed phase may be combined with a filler. Examples of fillers include, but are not limited to, talc, silica, calcium carbonates, micas, kaolin, zinc or titanium oxides, magnesium carbonates, silica silylate, titanium dioxide, glass or ceramic beads, polymethylmethacrylate beads, boron nitride, aluminum silicate, aluminum starch octenylsuccinate, bentonite, magnesium aluminum silicate, nylon, silk powder metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, silicone resin microbeads, and mixtures thereof. The fillers may be surface treated to modify affinity or compatibility with other ingredients.

### The Water Continuous Emulsion

Component B) is a water continuous emulsion that forms a first dispersed phase. Component B) may be a single water continuous emulsion, or a combination of water continuous emulsions. The water continuous emulsion (B) may be one or more than one water continuous emulsion which may be the same or different water continuous emulsions. The water continuous emulsion(s) useful as component B) in the present process contains at least one surfactant. The surfactant may vary, but typically is chosen from those surfactants that enhance the formation of water continuous emulsions. The surfactant may be an anionic surfactant, cationic surfactant, nonionic surfactant, amphoteric surfactant, or a mixture of any of these surfactants.

The first dispersed phase may comprise a silicone that is provided from a water continuous silicone emulsion containing at least one surfactant. The water continuous silicone emulsion containing at least one surfactant may be a single water continuous silicone emulsion, or a combination of water continuous silicone emulsions.

The water continuous silicone emulsions useful in the present bi-modal water continuous emulsions contain at least one surfactant. The surfactant may vary, but typically is chosen from those surfactants that enhance the formation of water continuous silicone emulsions.

The silicone in the water continuous silicone emulsion containing at least one surfactant may be any of those silicones listed above as hydrophobic oil A), and mixtures thereof.

The bi-modal water continuous emulsion may comprise a silicone formed by a hydrosilylation reaction and an amino-functionalized silicone. Such bi-modal water continuous emulsion can be made by first preparing an emulsion of Si-vinyl and Si—H functionalized polymers. Platinum catalyst is then added to perform the hydrosilylation cure inside the emulsion droplets. After the cure is complete, the amino-functionalized silicone is added to the emulsion in order to form a bi-modal water continuous emulsion.

Examples of anionic surfactants include, but are not limited to, alkali metal, amine, or ammonium salts of higher fatty acids, alkylaryl sulphonates such as sodium dodecyl benzene sulfonate, long chain fatty alcohol sulfates, olefin sulfates and olefin sulfonates, sulfated monoglycerides, sulfated esters, sulfonated ethoxylated alcohols, sulfosuccinates, alkane sulfonates, phosphate esters, alkyl isethionates, alkyl taurates, alkyl sarcosinates, and mixtures thereof.

Examples of cationic surfactants include, but are not limited to, alkylamine salts, quaternary ammonium salts, sulphonium salts, and phosphonium salts.

Examples of amphoteric surfactants include, but are not limited to, imidazoline compounds, alkylaminoacid salts, betaines, and mixtures thereof.

Examples of suitable nonionic surfactants include, but are not limited to, condensates of ethylene oxide with long chain fatty alcohols or fatty acids such as a C12-16 alcohol, condensates of ethylene oxide with an amine or an amide, condensation products of ethylene and propylene oxide, esters of glycerol, sucrose, sorbitol, fatty acid alkylol amides, sucrose esters, fluoro-surfactants, fatty amine oxides, and mixtures thereof. Further examples of nonionic surfactants include polyoxyethylene fatty alcohols such as polyoxyethylene (23) lauryl ether, polyoxyethylene (4) lauryl ether; ethoxylated alcohols such as ethoxylated trimethylnonanol, C₁₂-C₁₄ secondary alcohol ethoxylates, ethoxylated, C10-Guerbet alcohol, ethoxylated, iso-C13 alcohol; poly(oxyethylene)-poly(oxypropylene)-poly(oxyethylene) tri-block copolymer (also referred to as poloxamers); tetrafunctional poly(oxyethylene)-poly(oxypropylene) block copolymer derived from the sequential addition of propylene oxide and ethylene oxide to ethylene diamine (also referred to as poloxamines), silicone polyethers, and mixtures thereof.

When mixtures containing nonionic surfactants are used, one nonionic surfactant may have a low Hydrophile-Lipophile Balance (HLB) and the other nonionic surfactant(s) may have a high HLB, such that the nonionic surfactants have a combined HLB of 11-15, alternatively a combined HLB of 12.5-14.5.

Further examples of nonionic surfactants include polyoxyethylene alkyl ethers, polyoxyethylene alkylphenol ethers, polyoxyethylene lauryl ethers, straight-chain, primary alcohol alkoxylates, straight-chain secondary alcohol alkoxylates, alkyl phenol alkoxylates, olefinic alkoxylates, branched chain alkoxylates, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, polyoxyalkylene-substituted silicones (rake or ABn types), silicone alkanolamides, silicone esters, silicone glycosides, and mixtures thereof.

Further examples of nonionic surfactants include dimethicone copolyols, fatty acid esters of polyols, for instance sorbitol or glyceryl mono-, di-, tri- or sesquioleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers.

Further examples of anionic surfactants include carboxylates (sodium 2-(2-hydroxyalkyloxy)acetate)), amino acid derivatives (N-acylglutamates, N-acylglycinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Further examples of amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

The water continuous silicone emulsion (B) may be selected from those considered in the art to be a “macro” emulsion. In other words, the average volume particle size of the water continuous emulsion (B) may vary from 0.2 to 1000 μm, alternatively from 0.2 to 500 μm, alternatively from 0.2 to 100 μm, alternatively from 0.2 to 50 μm, alternatively from 0.2 to 30 μm, alternatively from 0.2 to 20 μm, alternatively from 0.2 to 10 μm, alternatively from 1 to 10 μm.

In some embodiment, the water continuous silicone emulsion (B) may be an emulsion having an average volume particle size of less than 200 nm.

The water continuous silicone emulsion (B) may be considered an “emulsion polymer”, in other words, an emulsion formed by emulsion polymerization techniques. Examples of suitable silicone emulsions produced by emulsion polymerization techniques are described in U.S. Pat. No. 2,891,920, U.S. Pat. No. 3,294,725, U.S. Pat. No. 5,661,215, U.S. Pat. No. 5,817,714, and U.S. Pat. No. 6,316,541.

The water continuous silicone emulsion (B) may be a mechanical emulsion. As used herein, mechanical emulsions refer to those emulsion in the art produced by using mechanical energy (such as from high shearing forces). Examples of silicone emulsions produced by mechanical techniques are described in U.S. Pat. No. 6,395,790.

The water continuous silicone emulsion (B) may be prepared using suspension polymerization techniques. Examples of silicone emulsions produced by suspension polymerization techniques are described in U.S. Pat. No. 4,618,645, U.S. Pat. No. 6,248,855, and U.S. Pat. No. 6,395,790.

Mixing in step I) can be accomplished by any method known in the art to effect mixing of high viscosity materials. The mixing may occur either as a batch, semi-continuous, or continuous process. Mixing may occur, for example using, batch mixing equipments with medium/low shear include change-can mixers, double-planetary mixers, conical-screw mixers, ribbon blenders, double-arm or sigma-blade mixers; batch equipments with high-shear and high-speed dispersers include those made by Charles Ross & Sons (NY), Hockmeyer Equipment Corp. (NJ); batch equipments with high shear actions include Banbury-type (CW Brabender Instruments Inc., NJ) and Henschel type (Henschel mixers America, TX). Illustrative examples of continuous mixers/compounders include extruders single-screw, twin-screw, and multi-screw extruders, co-rotating extruders, such as those manufactured by Krupp Werner & Pfleiderer Corp (Ramsey, N.J.), and Leistritz (NJ); twin-screw counter-rotating extruders, two-stage extruders, twin-rotor continuous mixers, dynamic or static mixers or combinations of these equipments.

The temperature and pressure at which the mixing of step I) occurs is not critical, but generally is conducted at ambient temperature and pressures. Typically, the temperature of the mixture will increase during the mixing process due to the mechanical energy associated when shearing such high viscosity materials.

Typically 1 to 1000 parts by weight of the water continuous emulsion (B) are mixed for every 100 parts by weight of component (A) in the step I) mixture, alternatively from 5 to 500 parts per 100 parts by weight of component (A) in the step I) mixture, or alternatively from 5 to 100 parts per 100 parts by weight of component (A) the step I) mixture.

Alternatively, the amount of component (A) may be from 20% to 80% by weight of the bi-modal water continuous emulsion, and the amount of component (B) may be from 20% to 80% by weight of the bi-modal water continuous emulsion.

In one embodiment, step I) may involve forming a mixture consisting essentially of 100 parts by weight of a hydrophobic oil (A), 1 to 1000 parts by weight of a water continuous emulsion (B) having at least one surfactant. In this embodiment, the mixture formed in step I) is essentially free from any additional surfactant compounds or components other than components (A) and (B). As used herein, “essentially free” means no additional surfactant compounds are added to the mixture formed in step I), other than the surfactant(s) present in the water continuous emulsion (B).

Step II) of the process involves admixing additional quantities of the water continuous emulsion (B) and/or water to the mixture from step I) to form a bi-modal water continuous emulsion. After steps I) and II) are completed, the first dispersed phase comprises component (A) and the second dispersed phase comprise component (B) such that the first dispersed phase and the second dispersed comprises at least 70 weight percent of the bi-modal water continuous emulsion.

The amount of the additional quantities of the water continuous emulsion (B) and/or water used in step II) may vary depending on the selection of components (A) and (B). Typically the amount of additional water continuous emulsion (B) and/or water admixed in step II) may vary from 1 to 1000 parts by weight of the step I) mixture, alternatively from 5 to 500 parts per 100 parts by weight, or alternatively from 5 to 100 parts per 100 parts by weight.

In step II), additional quantities of the water continuous emulsion (B) may be used alone, or alternatively be combined with varying quantities of water. Alternatively, additional quantities of water may be added alone without any additional quantities of the water continuous emulsion (B). The selection of using additional quantities of the water continuous emulsion (B) alone, in combination with varying amounts of water, or water alone will depend on the initial selection of the water continuous emulsion (B) and the desired physical properties of the resulting bi-modal water continuous emulsion. For example, high solids bi-modal water continuous emulsions may be prepared with only the addition of the water continuous emulsion (B). Conversely, low solids bi-modal water continuous emulsions may require the addition of water.

The water continuous emulsion (B) and/or water is added to the mixture from step I) at such a rate, with additional mixing, so as to form an emulsion of the mixture of step I). The water continuous emulsion (B) added to the mixture from step I) may be done in incremental portions, whereby each incremental portion comprises less than 50 weight % of the mixture from step I), alternatively 25 weight % of the mixture from step I), and each incremental portion of water continuous emulsion (B) is added successively to the previous after the dispersion of the previous incremental portion of water continuous emulsion (B), wherein sufficient incremental portions of water continuous emulsion (B) are added to form the bi-modal water continuous emulsion.

The number of incremental portions of the water continuous emulsion (B) and/or water added to the mixture from step I) may vary, but typically at least 2, alternatively, at least 3 incremental portions are added.

Mixing in step II) can be accomplished by any method known in the art to effect mixing of high viscosity materials and/or effect the formation of an emulsion. The mixing may occur either as a batch, semi-continuous, or continuous process. Any of the mixing methods as described for step I), may be used to effect mixing in step II). Alternatively, mixing in step II) may also occur via those techniques known in the art to provide high shear mixing to effect formation of emulsions. Representative of such high shear mixing techniques include high speed stirrers, homogenizers, Sonolators®, Microfluidizers®, Ross mixers, Eppenbach colloid mills, Flacktek Speedmixers®, and other similar shear devices.

Optionally, the bi-modal water continuous emulsion formed in step II) may be further sheared according to an optional step III) to reduce particle size and/or improve long term storage stability. The shearing may occur by any of the mixing techniques discussed above.

The bi-modal water continuous emulsions prepared according to the present invention may be characterized by a bi-modal particle size distribution. Particularly, the water continuous emulsion (B) forms a first dispersed phase of particle size P1 and the hydrophobic oil (A) forms a second dispersed phase of particle size P2, wherein the ratio P2:P1 is less than 1.

The particle size may be determined by laser diffraction of the emulsion. Suitable laser diffraction techniques are well known in the art. The particle size is obtained from a particle size distribution (PSD). The PSD can be determined on a volume, surface, length basis. The volume particle size is equal to the diameter of the sphere that has the same volume as a given particle. The term Dv, as used herein, represents the average volume particle size of the dispersed particles. Dv50 is the particle size measured in volume corresponding to 50% of the cumulative particle population. In other words, if Dv50=10 μm, 50% of the particle have an average volume particle size below 10 μm and 50% of the particle have a volume average particle size above 10 μm. Dv90 is the particle size measured in volume corresponding to 90% of the cumulative particle population. Mode 1 is the median of the distribution of one of the dispersed phase particle populations within a bi-modal particle size distribution and Mode 2 is the median of the other dispersed phase.

In some instances, it may be necessary to conduct two separate evaluations of particle size, especially when the particle sizes distributions of the resulting bi-modal water continuous emulsions exhibit a wide variation in size. In these instances a Malvern-Mastersizer® 2000 may be used to obtain particle size distributions in the range 0.5 to 1000 μm, while a Microtrac-Nanotrac® may be used to measure particle size distributions in the ranges less than 0.5 μm.

The average volume particle size of the dispersed particles in the bi-modal water continuous emulsions ranges from 0.001 μm to 1000 μm; or from 0.01μm to 20 μm; or from 0.02 μm to 10 μm.

Alternatively, the average volume particle size of each of the dispersed phases (that is, the first dispersed phase and the second dispersed phase) may be reported. The average volume particle size of the first dispersed phase of the bi-modal water continuous emulsions ranges from 0.1 μm to 500 μm; or from 0.1 μm to 100 μm; or from 0.2 μm to 30 μm. The average volume particle size of the second dispersed phase of the bi-modal water continuous emulsions ranges from 0.1 μm to 500 μm; or from 0.1 μm to 100 μm; or from 0.2 μm to 30 μm.

While not wishing to be bound by any theory, it is believed particle size distribution of the second dispersed phase results from the emulsification of the hydrophobic oil (A), while particle size distribution of the first dispersed phase results from the particles originating from the water continuous emulsion (B). However, there may be certain instances where the two particle size distribution overlap sufficiently that a bi-modal distribution may not be observable using the particle size determination techniques described above. The bi-modal particle size distribution may also be observed using optical microscopy techniques.

In one embodiment, the ratio P2:P1 is from 0.01 to 0.99, alternatively from 0.05 to 0.90, alternatively from 0.05 to 0.80, alternatively from 0.05 to 0.70, alternatively from 0.05 to 0.60, alternatively from 0.05 to 0.50, alternatively from 0.05 to 0.40, alternatively from 0.05 to 0.30, alternatively from 0.05 to 0.20, alternatively from 0.1 to 0.40, alternatively from 0.1 to 0.30, or alternatively from 0.1 to 0.20.

In one embodiment, the average volume particle size of the first dispersed phase (P1) of the bi-modal water continuous emulsions ranges from 1 μm to 20 μm and the average volume particle size of the second dispersed phase (P2) ranges from 0.1 μm to 5 μm wherein P1 is greater than P2. Alternatively, the average volume particle size of the first dispersed phase (P1) ranges from 1 μm to 10 μm and the average volume particle size the second dispersed phase (P2) ranges from 0.2 μm to 2.0 μm wherein P1 is greater than P2.

In some embodiments, there may be two, three or more modes in the resulting emulsion, such as to compose a bi-modal, tri-modal or multiple-modal emulsion.

In other embodiments, the bi-modal water continuous emulsions may be considered as a “high solids” emulsion, wherein the bi-modal water continuous emulsion contains at least 70% by weight of components (A) and (B), alternatively the bi-modal water continuous emulsion contains at least 75% by weight of components (A) and (B), alternatively the bi-modal water continuous emulsion contains at least 80% by weight of components (A) and (B), alternatively the bi-modal water continuous emulsion contains at least 85% by weight of components (A) and (B), or alternatively the bi-modal water continuous emulsion contains at least 90% by weight of components (A) and (B).

The “high solids” bi-modal water continuous emulsion may remain pourable. The bi-modal water continuous emulsion may have a viscosity from 10,000 to 1,000,000 mPa/s (10⁻³ Pa·s), alternatively 10,000 to 600,000 mPa/s (10⁻³ Pa·s), alternatively 12,000 to 600,000 mPa/s (10⁻³ Pa·s). In some embodiments, the bi-modal water continuous emulsion may have a viscosity less than 600,000 mPa/s (10⁻³ Pa·s), alternatively less than 200,000 mPa/s (10⁻³ Pa·s), or alternatively less than 100,000 mPa/s (10⁻³ Pa·s), as measured at 25° C.

The total surfactant concentration in the bi-modal water continuous emulsion may be from 0.01% to 20%, alternatively from 0.01% to 15%, alternatively from 0.01% to 10%, alternatively from 0.01% to 5%, or alternatively from 0.01% to 0.1% by weight. In some embodiment, the total surfactant concentration may be less than 20%, alternatively less than 15%, alternatively less than 10%, alternatively less than 5%, alternatively less than 1.0%, alternatively less than 0.2%, alternatively less than 0.1% by weight of the bi-modal water continuous emulsion.

In one embodiment, the bi-modal water continuous silicone emulsion produced according to the present invention may contain less than 1.0 weight % cyclosiloxanes, alternatively contains less than 0.5 weight % cyclosiloxanes, alternatively contains less than 0.1 weight % cyclosiloxanes. In another embodiment, the bi-modal water continuous silicone emulsion may contain less than 1.0 weight % of each octamethylcyclotetrasiloxanes (D₄) and decamethylcyclopentasiloxanes (D₅), alternatively contains less than 0.5 weight % of each octamethylcyclotetrasiloxanes (D₄) and decamethylcyclopentasiloxanes (D₅), or alternatively contains less than 0.1 weight % of each octamethylcyclotetrasiloxanes (D₄) and decamethylcyclopentasiloxanes (D₅).

The bi-modal water continuous emulsions of the present invention may contain additional components. Additional components may include solvents, diluents, or mixtures thereof. Solvents include low molecular weight organic solvents that are highly soluble in water, e.g., C1-C4 monohydric alcohols, C2-C5 polyhydric alcohols including alkylene glycols, polyalkylene glycols, alkylene carbonates, and mixtures thereof. Typical solvents include ethanol, propanol, isopropanol, n-butyl alcohol, t-butyl alcohol, ethylene glycol, diethylene glycol, propylene glycol, dipropylene glycol, glycerol, propylene carbonate, and mixtures thereof.

Further additional components may include, but are not limited to, color treatments, thickeners, water phase stabilizing agents, pH controlling agents, preservatives and biocides, pigments, colorants, dyes, soil release agents, oxidizing agents, reducing agents, inorganic salts, antibacterial agents, antifungal agents, bleaching agents, sequestering agents, and mixtures thereof.

Examples of thickeners include, but are not limited to, acrylamide polymers and copolymers, acrylate copolymers and salts thereof (such as sodium polyacrylate), xanthan gum and derivatives, cellulose gum and cellulose derivatives (such as methylcellulose, methylhydroxypropylcellulose, hydroxypropylcellulose, polypropylhydroxyethylcellulose), starch and starch derivatives (such as hydroxyethylamylose and starch amylase), polyoxyethylene, carbomer, hectorite and hectorite derivatives, sodium alginate, arabic gum, cassia gum, guar gum and guar gum derivatives, cocamide derivatives, alkyl alcohols, gelatin, PEG-derivatives, saccharides (such as fructose, glucose) and saccharides derivatives (such as PEG-120 methyl glucose diolate), and mixtures thereof.

Examples of water phase stabilizing agents include, but are not limited to, electrolytes (e.g. alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate), polyols (glycerine, propylene glycol, butylene glycol, and sorbitol), alcohols such as ethyl alcohol, and hydrocolloids, and mixtures thereof.

Examples of pH controlling agents, but are not limited to, include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, citric acid, and mixtures thereof.

Example of preservatives and biocides include, but are not limited to, paraben derivatives, hydantoin derivatives, chlorhexidine and its derivatives, imidazolidinyl urea, phenoxyethanol, silver derivatives, salicylate derivatives, triclosan, ciclopirox olamine, hexamidine, oxyquinoline and its derivatives, PVP-iodine, zinc salts and derivatives such as zinc pyrithione, glutaraldehyde, formaldehyde, 2-bromo-2-nitropropane-1,3-diol, 5-chloro-2-methyl-4-isothiazoline-3-one, 2-methyl-4-isothiazoline-3-one, and mixtures thereof.

Examples of pigments and colorants include, but are not limited to, surface treated or untreated iron oxides, surface treated or untreated titanium dioxide, surface treated or untreated mica, silver oxide, silicates, chromium oxides, carotenoids, carbon black, ultramarines, chlorophyllin derivatives and yellow ocher. Examples of organic pigments include, but are not limited to, aromatic types including azo, indigoid, triphenylmethane, anthraquinone, and xanthine dyes which are designated as D&C and FD&C blues, browns, greens, oranges, reds, yellows, etc, and mixtures thereof. Surface treatments include those treatments based on lecithin, silicone, silanes, fluoro compounds.

A dye may generally be described as a coloured substance that has an affinity to the substrate to which it is being applied. Examples of dyes include, but are not limited to, anionic dyes (for example a direct dye or an acid dye), reactive dyes, nonionic dyes (for example a disperse dye) or pigment dyes (for example a vat dye).

Examples of soil release agents include, but are not limited to, copolymeric blocks of terephthalate and polyethylene oxide or polypropylene oxide, and the like.

Examples of oxidizing agents include, but are not limited to, ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, and mixtures thereof.

Examples of reducing agents include, but are not limited to, ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, and mixtures thereof.

Non-limiting examples of suitable inorganic salts include: MgI₂, MgBr₂, MgCl₂, Mg(NO₃)₂, Mg₃(PO₄)₂, Mg₂P₂O₇, MgSO₄, magnesium silicate, NaI, NaBr, NaCl, NaF, Na₃(PO₄), NaSO₃, Na₂SO₄, Na₂SO₃, NaNO₃, NaIO₃, Na₃(PO₄), Na₄P₂O₇, sodium silicate, sodium metasilicate, sodium tetrachloroaluminate, sodium tripolyphosphate (STPP), Na₂Si₃O₇, sodium zirconate, CaF₂, CaCl₂, CaBr₂, CaI₂, CaSO₄, Ca(NO₃)₂, Ca, KI, KBr, KCl, KF, KNO₃, KIO₃, K₂SO₄, K₂SO₃, K₃(PO₄), K₄(P₂O₇), potassium pyrosulfate, potassium pyrosulfite, LiI, LiBr, LiCl, LiF, LiNO₃, AlF₃, AlCl₃, AlBr₃, AlI₃, Al₂(SO₄)₃, Al(PO₄), Al(NO₃)₃, aluminum silicate; including hydrates of these salts and including combinations of these salts or salts with mixed cations e.g. potassium alum AIK(SO₄)₂ and salts with mixed anions, e.g. potassium tetrachloroaluminate and sodium tetrafluoroaluminate. Salts incorporating cations from groups IIIa, IVa, Va, VIa, VIIa, VIII, Ib, and IIb on the periodic chart with atomic numbers>13 are also useful in reducing dilution. Salts with cations from group Ia or IIa with atomic numbers>20 as well as salts with cations from the lactinide or actinide series are useful in reducing dilution viscosity, and mixtures thereof.

Examples of antibacterial agents include, but are not limited to, chlorohexadiene gluconate, alcohol, benzalkonium chloride, benzethonium chloride, hydrogen peroxide, methylbenzethonium chloride, phenol, poloxamer 188, povidone-iodine, and mixtures thereof.

Examples of antifungal agents include, but are not limited to, miconazole nitrate, calcium undecylenate, undecylenic acid, zinc undecylenate, and mixtures thereof.

Examples of bleaching agents include, but are not limited to, chlorine bleaches such as chlorine, chlorine dioxide, sodium hypochlorite, calcium hypochlorite, sodium chlorate; peroxide bleaches such as hydrogen peroxide, sodium percarbonate, sodium perborate; reducing bleaches such as sodium dithionite, sodium borohydride; ozone; and mixtures thereof.

Examples of sequestering agents (also chealting agents) include, but are not limited to, phosphonates; amino carboxylic acid compounds (such as ethylenediamine tetraacetic acid (EDTA); N-hydroxyethylenediamine triacetic acid; nitrilotriacetic acid (NTA); and diethylenetriamine pentaacetic acid (DEPTA)); organo am inophosphonic acid compounds (such as ethylenediamine tetrakis (methylenephosphonic acid); 1-hydroxyethane 1,1-diphosphonic acid (HEDP); and aminotri (methylenephosphonic acid)); and mixtures thereof

Examples of diluents include, but are not limited to, silicon containing diluents such as hexamethyldisiloxane, octamethyltrisiloxane, and other short chain linear siloxanes such as octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, cyclic siloxanes such as hexamethylcyclotrisiloxane, octamethylcyclotetrasiloxane, decamethylcyclopentasiloxane, dodecamethylcyclohexasiloxane; organic diluents such as butyl acetate, alkanes, alcohols, ketones, esters, ethers, glycols, glycol ethers, hydrofluorocarbons or any other material which can dilute the formulation without adversely affecting any of the component materials of the cosmetic composition. Hydrocarbons include, but are not limited to, isododecane, isohexadecane, Isopar L (C11-C13), Isopar H (C11-C12), hydrogenated polydecene. Ethers and esters include, but are not limited to, isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, and octyl palmitate. Additional organic diluents include, but are not limited to, fats, oils, fatty acids, and fatty alcohols.

The bi-modal water continuous emulsions of the present invention are useful for a variety of applications where it is desirable to provide pourable water-based organic or silicone materials having a high solids content. Such applications include various coating applications, fabric care applications, and fabric softening applications. The bi-modal water continuous emulsions of the present invention may also be used or beneficial in personal care applications.

The personal care compositions may be in the form of a cream, a gel, a powder, a paste, or a freely pourable liquid. Generally, such compositions can generally be prepared at room temperature if no solid materials at room temperature are present in the compositions, using simple propeller mixers, Brookfield counter-rotating mixers, or homogenizing mixers. No special equipment or processing conditions are typically required. Depending on the type of form made, the method of preparation will be different, but such methods are well known in the art.

The personal care compositions may be functional with respect to the portion of the body to which it is applied, cosmetic, therapeutic, or some combination thereof. The personal care compositions may be used for application to skin or hair. Conventional examples of such personal care products include, but are not limited to, antiperspirants and deodorants, skin care creams, skin care lotions, moisturizers, facial treatments such as acne or wrinkle removers, personal and facial cleansers, bath oils, perfumes, colognes, sachets, sunscreens, pre-shave and after-shave lotions, shaving soaps, and shaving lathers, hair shampoos, hair conditioners, hair colorants, hair relaxants, hair sprays, mousses, gels, permanents, depilatories, and cuticle coats, make-ups, color cosmetics, foundations, concealers, blushes, lipsticks, eyeliners, mascara, oil removers, color cosmetic removers, and powders, medicament creams, pastes or sprays including anti-acne, dental hygienic, antibiotic, healing promotive, nutritive and the like, which may be preventative and/or therapeutic. In general, the personal care composition may be formulated with a carrier that permits application in any conventional form, including but not limited to liquids, rinses, lotions, creams, pastes, gels, foams, mousses, ointments, sprays, aerosols, soaps, sticks, soft solids, solid gels, and gels. Suitable carriers are appreciated in the art.

The personal care composition can be used in or for a variety of personal, household, and healthcare applications. In particular, the bi-modal water continuous emulsions may be used in the personal care products as described in U.S. Pat. Nos. 6,051,216, 5,919,441, 5,981,680; WO 2004/060271 and WO 2004/060101; in sunscreen compositions as described in WO 2004/060276; in cosmetic compositions also containing film-forming resins, as described in WO 03/105801; in the cosmetic compositions as described in US Pat. App. Pub. Nos. 2003/0235553, 2003/0072730 and 2003/0170188, in EP Pat. Nos. 1,266,647, 1,266,648, and 1,266,653, in WO 03/105789, WO 2004/000247 and WO 03/106614; as additional agents to those described in WO 2004/054523; in long wearing cosmetic compositions as described in US Pat. App. Pub. No. 2004/0180032; and/or in transparent or translucent care and/or make up compositions as described in WO 2004/054524, all of which are expressly incorporated herein by reference in various non-limiting embodiments.

Non-limiting examples of additives which may be formulated into the personal care compositions include, but are not limited to, additional silicones, anti-oxidants, cleansing agents, colorants, additional conditioning agents, deposition agents, electrolytes, emollients and oils, exfoliating agents, foam boosting agents, solvents, suspending agents, fragrances, humectants, occlusive agents, pediculicides, pH control agents, pigments, preservatives, biocides, other solvents, stabilizers, sun-screening agents, suspending agents, tanning agents, other surfactants, thickeners, powder (that may include organic and/or inorganic pigments), inorganic or organic fillers, vitamins, botanicals, waxes, rheology-modifying agents, anti-dandruff, anti-acne, anti-carie and wound healing-promotion agents.

The personal care compositions, such as a shampoo or cleanser, may include at least one anionic detersive surfactant. This can be any of the well-known anionic detersive surfactants typically used in shampoo formulations. These anionic detersive surfactants can function as cleansing agents and foaming agents in the shampoo compositions. The anionic detersive surfactants are exemplified by alkali metal sulforicinates, sulfonated glyceryl esters of fatty acids such as sulfonated monoglycerides of coconut oil acids, salts of sulfonated monovalent alcohol esters such as sodium oleylisethianate, amides of amino sulfonic acids such as the sodium salt of oleyl methyl tauride, sulfonated products of fatty acids nitriles such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanol amine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, alkylarylsulfonates having 1 or more alkyl groups of 8 or more carbon atoms, alkylbenzenesulfonic acid alkali metal salts exemplified by hexylbenzenesulfonic acid sodium salt, octylbenzenesulfonic acid sodium salt, decylbenzenesulfonic acid sodium salt, dodecylbenzenesulfonic acid sodium salt, cetylbenzenesulfonic acid sodium salt, and myristylbenzenesulfonic acid sodium salt, sulfuric esters of polyoxyethylene alkyl ether including CH₃(CH₂)₆CH₂O(C₂H₄O)₂SO₃H, CH₃(CH₂)₇CH₂O(C₂H₄O)_{3.5}SO₃H, CH₃(CH₂)₈CH₂O(C₂H4O)₈SO₃H, CH₃(CH₂)₁₉CH₂O(C₂H₄O)₄SO₃H, and CH₃(CH₂)₁₀CH₂O(C₂H₄O)₆SO₃H, sodium salts, potassium salts, and amine salts of alkylnaphthylsulfonic acid. Typically, the detersive surfactant is chosen from sodium lauryl sulfate, ammonium lauryl sulfate, triethanolamine lauryl sulfate, sodium lauryl ether sulfate, and ammonium lauryl ether sulfate. The anionic detersive surfactant can be present in the shampoo composition in an amount from 5 to 50 weight percent and typically 5 to 25 weight percent based on the total weight of the shampoo composition.

The personal care compositions may include at least one cationic deposition aid, typically a cationic deposition polymer. The cationic deposition aid is typically present at levels of from 0.001% to 5%, typically from 0.01% to 1%, more typically from 0.02% to 0.5% by weight of the composition. The cationic deposition polymer may be a homopolymer or be formed from two or more types of monomers. The molecular weight of the cationic deposition polymer is typically from 5,000 to 10,000,000, typically at least 10,000 and typically from 100,000 to 2,000,000. The cationic deposition polymers typically have cationic nitrogen containing groups such as quaternary ammonium or protonated amino groups, or a combination thereof. The cationic charge density has been found to need to be at least 0.1 meq/g, typically above 0.8 or higher. The cationic charge density should not exceed 4 meq/g, it is typically less than 3 and more typically less than 2 meq/g. The charge density can be measured using the Kjeldahl method and is within the above limits at the desired pH of use, which will in general be from 3 to 9 and typically from 4 to 8. It is contemplated that any and all values or ranges of values between those described above may also be utilized. The cationic nitrogen-containing group is typically present as a substituent on a fraction of the total monomer units of the cationic deposition polymer. Thus when the cationic deposition polymer is not a homopolymer it can include spacer noncationic monomer units. Such cationic deposition polymers are described in the CTFA Cosmetic Ingredient Directory, 3rd edition, which is expressly incorporated herein by reference in one or more non-limiting embodiments. Suitable cationic deposition aids include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers typically have C1-C7 alkyl groups, more typically C1-C3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are typical. Amine substituted vinyl monomers and amines can be polymerized in the amine form and then converted to ammonium by quaternization. Suitable cationic amino and quaternary ammonium monomers include, for example, vinyl compounds substituted with dialkyl aminoalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g. alkyl vinyl imidazolium, and quaternized pyrrolidine, e.g. alkyl vinyl imidazolium, alkyl vinyl pyridinium, alkyl vinyl pyrrolidine salts. The alkyl portions of these monomers are typically lower alkyls such as the C1-C7 alkyls, more typically C1 and C2 alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are typically C1-C7 hydrocarbyls, more typically C1-C3, alkyls. The cationic deposition aids can include combinations of monomer units derived from amine- and/or quaternary ammonium-substituted monomer and/or compatible spacer monomers. Suitable cationic deposition aids include, for example: copolymers of 1-vinyl-2-pyrrolidine and 1-vinyl-3-methylimidazolium salt (e.g. Chloride salt) (referred to in the industry by the Cosmetic, Toiletry, and Fragrance Association, “CTFA” as Polyquaternium-16) such as those commercially available from BASF Wyandotte Corp. (Parsippany, N.J., USA) under the LUVIQUAT tradename (e.g. LUVIQUAT FC 370); copolymers of 1-vinyl-2-pyrrolidine and dimethylaminoethyl methacrylate (referred to in the industry by CTFA as Polyquaternium-11) such as those commercially from Gar Corporation (Wayne, N.J., USA) under the GAFQUAT tradename (e.g. GAFQUAT 755N); cationic diallyl quaternary ammonium-containing polymer including, for example, dimethyl diallyammonium chloride homopolymer and copolymers of acrylamide and dimethyl diallyammonium chloride, referred to in the industry (CTFA) as Polyquaternium 6 and Polyquaternium 7, respectively; mineral acid salts of aminoalkyl esters of homo- and co-polymers of unsaturated carboxylic acids having from 3 to 5 carbon atoms, as described in U.S. Pat. No. 4,009,256; and cationic polyacrylamides as described in UK Application No. 9403156.4 (WO95/22311), each of which is expressly incorporated herein in one or more non-limiting embodiments.

Other cationic deposition aids that can be used include polysaccharide polymers, such as cationic cellulose derivatives and cationic starch derivatives. Cationic polysaccharide polymer materials suitable for use in compositions of the disclosure include those of the formula: A-O(R—N+R¹R²R³x⁻) wherein: A is an anhydroglucose residual group, such as starch or cellulose anhydroglucose residual, R is an alkylene oxyalklene, polyoxyalkylene, or hydroxyalkylene group, or combination thereof, R¹, R² and R³ independently are alkyl, aryl, alkylaryl, arylalkyl, alkoxyalkyl, or alkoxyaryl groups, each group containing up to 18 carbon atoms, and the total number of carbon atoms for each cationic moiety (i.e., the sum of carbon atoms in R¹, R², R³) typically being 20 or less, and X is an anionic counterion, as previously described. Cationic cellulose is available from Amerchol Corp. (Edison, N.J., USA) in their Polymer iR (Trademark) and LR (Trademark) series of polymers, as salts of hydroxyethyl cellulose reacted with trimethyl ammonium substituted epoxide, referred to in the industry (CTFA) as Polyquaternium 10.

Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200. Other cationic deposition aids that can be used include cationic guar gum derivatives, such as guar hydroxypropyltrimonium chloride (Commercially available from Celanese Corp. in their Jaguar Trademark series). Other materials include quaternary nitrogen-containing cellulose ethers (e.g. as described in U.S. Pat. No. 3,962,418), and copolymers of etherified cellulose and starch (e.g. as described in U.S. Pat. No. 3,958,581), each of which is expressly incorporated herein by reference in one or more non-limiting embodiments.

Having described the invention with reference to certain embodiments, other embodiments will become apparent to one skilled in the art from consideration of the specification. The invention is further defined by reference to the following examples describing the preparation of the emulsions and processes of the invention. It will be apparent to those skilled in the art that many modifications, both to materials and procedures, may be practiced without departing from the scope of the invention.

### EXAMPLES

The following examples are included to illustrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in weight percent. All measurements were conducted at 23° C. unless indicated otherwise.

### Example 1

### Bimodal Emulsion of PDMS and ABn Copolymer.

A large particle size emulsion, hereafter referred to as base Emulsion 1 was prepared. 60.28 g of 60,000 cSt polydimethylsiloxane (PDMS) was added to a Max 100 dental cup. 0.87 g of Brij 30 (also called Brij L4), 2.70 g of Brij 35L (also called Brij L23-69-LQ-AP), and 7.80 g of water were added to the dental cup. The cup contents were mixed on the DAC 150 mixer at 3500 rpm for 30 seconds. After mixing, the emulsion phase inverted to an oil-in-water emulsion. More water was then added to dilute the emulsion: 4.97 g water was added and mixed on DAC 150 at 3500 rpm for 30 seconds; 4.98 g water was added and mixed on DAC 150 at 3500 rpm for 30 seconds. The particle size of the emulsion was measured on the Malvern Mastersizer 2000. The DV50 particle size was 6.726 μm, and the DV90 particle size was 12.013 μm.

After preparing base Emulsion 1, bis-diisopropanolamino-PG-propyl dimethicone/bis-isobutyl PEG-14 copolymer was added to form a bimodal water continuous emulsion. 6.30 g of bis-diisopropanolamino-PG-propyl dimethicone/bis-isobutyl PEG-14 copolymer and 6.01 g of base Emulsion 1 were added to a Max 20 dental cup. The cup contents were mixed on the DAC 150 at 3500 rpm for 30 seconds. More of base Emulsion 1 was added in order to dilute the emulsion. 1.95 g of base Emulsion 1 was added and mixed on DAC 150 at 3500 rpm for 30 seconds. 2.00 g of base Emulsion 1 was added and mixed on DAC 150 at 3500 rpm for 30 seconds. The particle size was measured on the Malvern Mastersizer 2000. There are particle size modes at 1.011 μm and 6.575 μm.

### Example 2

### Bimodal Emulsion of High Molecular Weight Hydrosilylation Product and ABn Copolymer

A large particle size emulsion was prepared and referred to as base Emulsion 2. 52.73 g of 55,000 cSt PDMS, dimethylvinylsiloxy-terminated and 1.08 g of hydrogen-terminated PDMS, with the average structure H(Me₂SiO)₁₅SiMe₂H where Me denotes the methyl radical were added to a Max 100 dental cup and mixed on the DAC 150 at 3500 rpm for 30 seconds. 2.10 of Brij L23-69-LQ-AP, 1.85 g of Brij L4, and 10.04 g of water were added to the dental cup. Mixing was on the DAC 150 at 3500 rpm for 30 seconds. After mixing, there were some small agglomerates of unemulsified silicone in some instances. Mixing was continued on the DAC 150 until the silicone was fully emulsified.

The hydrosilylation reaction was catalyzed using a Karstedt-type catalyst with 20-25% of the platinum as elemental platinum. 1.35% active solution of the Karstedt catalyst in 1.5 cSt. PDMS was prepared. This was done by adding the catalyst to a glass jar and shaking by hand. After preparing the solution, 0.10 g of the solution was added to base Emulsion 2. The emulsion was allowed to cure. After the cure, the emulsion was further diluted with water to reduce the bulk viscosity if so desired, for example, 0.98 g, 1.03 g, 2.00 g, and 4.01 g of water were added consecutively to base Emulsion 2 and mixed on the DAC 150 at 3500 rpm for 30 seconds following each addition. After dilution, the internal phase polymer was separated using isopropanol solvent. The internal phase viscosity was >100M cP. Base Emulsion 2 was measured on the ARES rheometer as having an in-phase viscosity of 361 million cP at 0.01 Hz. The emulsion particle size was measured on the Malvern Mastersizer 2000. The DV50 particle size was 5.902 μm, and the DV90 particle size was 10.283 μm.

After preparing base Emulsion 2, bis-diisopropanolamino-PG-propyl dimethicone/bis-isobutyl PEG-14 copolymer was added to form a bimodal water continuous emulsion. 12.43 g of bis-diisopropanolamino-PG-propyl dimethicone/bis-isobutyl PEG-14 copolymer and 12.06 g of base Emulsion 2 were added to a Max 40 dental cup. The cup contents are mixed on the DAC 150 at 3500 rpm for 30 seconds. More of base Emulsion 2 was added in order to dilute the emulsion. 5.21 g of base Emulsion 2 was added and mixed on DAC 150 at 3500 rpm for 30 seconds. The particle size was measured on the Malvern Mastersizer 2000. There were particle size modes at 0.752 μm and 7.065 μm.

### Example 3

### Bimodal Emulsion of High Molecular Weight Hydrosilylation Product and Amodimethicone

This example uses base Emulsion 2, which is described in Example 2. After preparing base Emulsion 2, a silicone polymer with amino functionality is added to form a bi-modal water continuous emulsion. 6.23 g of amodimethicone and 6.00 g of base Emulsion 2 were added to a Max 20 dental cup. The cup contents were mixed on the DAC 150 at 3500 rpm for 30 seconds. More of base Emulsion 2 was added in order to dilute the emulsion. 4.21 g of base Emulsion 2 was added and mixed on DAC 150 at 3500 rpm for 30 seconds. The particle size was measured on the Malvern Mastersizer 2000. There were particle size modes at 1.123 μm and 6.172 μm.

What is claimed is:

1. A process for making a bi-modal water continuous emulsion comprising:
I) forming a mixture comprising:
A) 100 parts by weight of a hydrophobic oil, and
B) 1 to 1000 part by weight of a water continuous emulsion having at least one surfactant;
II) admixing additional quantities of the water continuous emulsion and/or water to the mixture from step I) to form a bi-modal water continuous emulsion,
wherein the water continuous emulsion forms a first dispersed phase of particle size P1 and the hydrophobic oil forms a second dispersed phase of particle size P2, and wherein the ratio P2:P1 is less than 1.

2. The process of claim 1, wherein the mixture of step I) consists essentially of components A) and B).

3. The process of claim 1, wherein the first dispersed phase and the second dispersed phase comprise at least 70 weight percent of the bi-modal water continuous emulsion.

4. The process of claim 1, wherein the hydrophobic oil is a silicone or organic oil.

5. The process of claim 4, wherein the silicone is an amino-functionalized silicone.

6. The process of claim 4, wherein the silicone is an organopolysiloxanes having amino and polyether functionalities.

7. The process of claim 4, wherein the silicone is an amino-terminated organopolysiloxanes-polyether block copolymer.

8. The process of claims 1, wherein the water continuous emulsion is a silicone emulsion or an organic emulsion.

9. The process of 8, wherein silicone emulsion comprises a silicone that is a product of a hydrosilylation reaction.

10. The process of claim 1, wherein the quantity of the water continuous emulsion and/or water added to the mixture is such so as to provide a bi-modal water continuous emulsion containing at least 70% by weight of components A) and B).

11. The process of claim 1, wherein the bi-modal water continuous emulsion has a viscosity of less than 100,000 cP.

12. The process of claim 1, wherein the surfactant concentration in the bi-modal water continuous emulsion is from 0.01% to 20% by weight

13. The process of claim 1, wherein the bi-modal water continuous emulsion contains less than 1% by weight of cyclosiloxanes.

14. The process of claim 1, wherein the ratio P2:P1 is from 0.01 to 0.99, from 0.05 to 0.80, from 0.05 to 0.50, from 0.05 to 0.20, or from 0.1 to 0.2.

15. A bi-modal water continuous emulsion produced by the process of claims 1.

16. A bi-modal water continuous emulsion, wherein the emulsion comprises an hydrophobic oil as the first dispersed phase and an amino-functionalized silicone as the second dispersed phase, wherein the first dispersed phase has a particle size P1 and the second dispersed phase has a particle size P2, and wherein the ratio P2:P1 is less than 1.

17. The bi-modal water continuous emulsion of claim 16, wherein the hydrophobic oil is silicone that is a product of a hydrosilylation reaction.

18. The bi-modal water continuous emulsion of claim 16, wherein the amino-functionalized silicone is an organopolysiloxanes having amino and polyether functionalities.

19. A personal care composition comprising the bi-modal water continuous emulsion of claim 16.

20. A coating composition comprising the bi-modal water continuous emulsion of claim 16.

What is claimed is:

1. A process for making a bi-modal water continuous emulsion comprising:
I) forming a mixture comprising:
A) 100 parts by weight of a hydrophobic oil, and
B) 1 to 1000 part by weight of a water continuous emulsion having at least one surfactant;
II) admixing additional quantities of the water continuous emulsion and/or water to the mixture from step I) to form a bi-modal water continuous emulsion,
wherein the water continuous emulsion forms a first dispersed phase of particle size P1 and the hydrophobic oil forms a second dispersed phase of particle size P2, and wherein the ratio P2:P1 is less than 1.

2. The process of claim 1, wherein the mixture of step I) consists essentially of components A) and B).

3. The process of claim 1 or 2, wherein the first dispersed phase and the second dispersed phase comprise at least 70 weight percent of the bi-modal water continuous emulsion.

4. The process of claim 1 or 2, wherein the hydrophobic oil is a silicone or organic oil.

5. The process of claim 4, wherein the silicone is an amino-functionalized silicone.

6. The process of claim 4, wherein the silicone is an organopolysiloxanes having amino and polyether functionalities.

7. The process of claim 6, wherein the silicone is an amino-terminated organopolysiloxanes-polyether block copolymer.

8. The process of any one of claims 1 to 7, wherein the water continuous emulsion is a silicone emulsion or an organic emulsion.

9. The process of 8, wherein silicone emulsion comprises a silicone that is a product of a hydrosilylation reaction.

10. The process of any one of the preceding claims, wherein the quantity of the water continuous emulsion and/or water added to the mixture is such so as to provide a bi-modal water continuous emulsion containing at least 70% by weight of components A) and B).

11. The process of any one of the preceding claims, wherein the bi-modal water continuous emulsion has a viscosity of less than 100,000 cP.

12. The process of any one of the preceding claims, wherein the surfactant concentration in the bi-modal water continuous emulsion is from 0.01% to 20% by weight

13. The process of any one of the preceding claims, wherein the bi-modal water continuous emulsion contains less than 1% by weight of cyclosiloxanes.

14. The process of any one of the preceding claims, wherein the ratio P2:P1 is from 0.01 to 0.99, from 0.05 to 0.80, from 0.05 to 0.50, from 0.05 to 0.20, or from 0.1 to 0.2.

15. A bi-modal water continuous emulsion produced by any one of the preceding claims.

16. A bi-modal water continuous emulsion, wherein the emulsion comprises an hydrophobic oil as the first dispersed phase and an amino-functionalized silicone as the second dispersed phase, wherein the first dispersed phase has a particle size P1 and the second dispersed phase has a particle size P2, and wherein the ratio P2:P1 is less than 1.

17. The bi-modal water continuous emulsion of claim 14, wherein the hydrophobic oil is silicone that is a product of a hydrosilylation reaction.

18. The bi-modal water continuous emulsion of claim 16 or 17, wherein the amino-functionalized silicone is an organopolysiloxanes having amino and polyether functionalities.

19. A personal care composition comprising the bi-modal water continuous emulsion of any of the preceding claims.

20. A coating composition comprising the bi-modal water continuous emulsion of any of the preceding claims.
